(19) United States
(12) Patent Application Publication
(10) Pub. No.: US 2025/0009922 A1
(43) Pub. Date: Jan. 9, 2025

(54) SYSTEMS AND METHODS FOR TARGETED SANITIZATION

(71) Applicant: SHYLD Al, INC., Santa Clara, CA (US)

(72) Inventors: Morteza Noshad, Santa Clara, CA (US); Mohammad Noshad, Santa Clara, CA (US)

(73) Assignee: SHYLD Al, INC., Santa Clara, CA (US)

(21) Appl. No.: 18/706,379

(22) PCT Filed: Dec. 30, 2022
(86) PCT No.: PCT/US2022/054371

§ 371 Date: Apr. 30, 2024

Related U.S. Application Data
(60) Provisional application No. 63/274,529, filed on Nov. 2, 2021.

(51) Int. Cl. A61L 2/24; A61L 2/08; A61L 2/10; A61L 2/26; G06V 20/69; G06V 40/20

(52) U.S. Cl. CPC A61L 2/24; A61L 2/084; A61L 2/085; A61L 2/10; A61L 2/26; A61L 2202/11; A61L 2202/14; A61L 2202/25; G06V 20/69; G06V 40/28

(57) ABSTRACT

Sanitizing a surface, according to the systems, the methods, the computer-readable media, or the techniques described herein, may include obtaining sensor data of the surface; predicting that one or more contaminants are deposited on the surface based at least in part on the sensor data of the surface; and based at least in part on predicting that the one or more contaminants are deposited on the surface, causing a light beam to be steered towards the surface, thereby sanitizing the surface of the one or more contaminants.

### CROSS-REFERENCE

This application claims priority to U.S. Provisional Application No. 63/274,529, filed Nov. 2, 2021, which is entirely incorporated herein by reference.

### BACKGROUND

New viruses and bacteria are discovered daily, many of which produce illnesses or other conditions. Further, some of these viruses and bacteria may have no known cure or vaccine. Infected people may spread viruses and bacteria even before they show symptoms of illness. Some of these viruses and bacteria can remain in the air and on surfaces for hours, which can make them highly contagious and difficult to contain. These viruses and bacteria may be particularly problematic when in public spaces that are confined such as hospitals, airports, offices, stores, etc. Controlling the spread of these viruses and bacteria (e.g., by reducing transmission probability from person to person) may buy time until a cure or a vaccine is developed.

Ultraviolet germicidal irradiation (UVGI) is a disinfection method that uses short-wavelength ultraviolet (UV-C) light to kill or inactivate these viruses and bacteria (e.g., microorganisms) by destroying nucleic acids and disrupting their DNA, leaving them unable to perform vital cellular functions. UVGI may be an effective disinfection technique in a variety of applications, such as those in healthcare, the food industry, air conditioning, and water purification. The effectiveness of germicidal UV radiation may depend on the length of time the viruses and bacteria are exposed to the UV radiation, the intensity of the UV radiation, or the wavelength of the UV radiation. But UV-C light may negatively impact human health and may thus be hazardous to use in public spaces. Current UV-C systems may transmit light in all directions in an area, where any object or human in the area is subject to radiation.

### SUMMARY

Effective prevention of infection from contaminants (e.g., pathogens, bacteria, viruses, microorganisms, infectious diseases, etc.) may benefit from smart and interactive approaches that sanitize (e.g., disinfect) air and surfaces that may be contaminated before people interact with the air or surfaces. In some embodiments, systems, methods, computer-readable media, and techniques for ultraviolet (UV) sanitization that use artificial intelligence (AI) to actively sanitize air and surfaces as people and objects move around may be provided.

In some embodiments, the systems, the methods, the computer-readable media, or the techniques may sanitize the surfaces (e.g., areas, objects, etc.) impacted by contact (e.g., movements, actions, etc.) of people. The AI engine may assign a score for the contamination level of each surface and adjust the sanitization time and UV light strength on each surface based on those scores. In some embodiments, more than one UV beam may be steered and focused on one or more surfaces at the same time. In some embodiments, the systems, the methods, the computer-readable media, or the techniques may be to achieve an interactive and real-time sanitization as people and other objects move through a space. In some embodiments, the systems, the methods, the computer-readable media, or the techniques may avoid exposing people to UV radiation by employing various sensors such as thermal sensors and imagers, infrared sensors and imagers, cameras, laser ranging sensors, microphones, or radar sensors or imagers. In some embodiments, the AI engine processes and augments data to detect human presence and avoids directing the UV light towards those areas. In some embodiments, the systems, the methods, the computer-readable media, or the techniques may can significantly reduce the person-to-person spread of viruses, bacteria, or other infectious pathogens in a highly efficient manner and slow down the spread of highly contagious microorganisms.

In some aspects, the present disclosure provides a method for sanitizing a surface, comprising: (a) obtaining, at one or more processors, sensor data of the surface; (b) predicting, by the one or more processors, that one or more contaminants are deposited on the surface based at least in part on the sensor data of the surface; and (c) based at least in part on predicting that the one or more contaminants are deposited on the surface at (b), causing, by the one or more processors, a light beam to be steered towards the surface, thereby sanitizing the surface of the one or more contaminants.

In some aspects, the present disclosure provides a method for sanitizing a surface in a confined location, comprising: (a) tracking, by one or more processors, a subject in the confined location to predict that the subject has deposited one or more contaminants on the surface in the confined location; and (b) based at least in part on predicting that the subject has deposited the one or more contaminants on the surface in the confined location, causing, by the one or more processors, a light beam to be steered towards the surface.

In some aspects, the present disclosure provides a system for sanitizing a surface in a confined location, comprising: (a) a light source configured to provide a light beam having a wavelength or wavelength range sufficient to sanitize the surface; (b) one or more sensors configured to track a subject in the confined location; and (c) one or more computer processors operatively coupled to the light source and the one or more sensors. The one or more computer processors are individually or collectively configured to: (i) predict, based at least in part on sensor data from the one or more sensors, that the subject has deposited one or more contaminants on the surface in the confined location; and (ii) based at least in part on predicting that the subject has deposited the one or more contaminants on the surface in the confined location, cause the light source to be steered to direct the light beam towards the surface.

Additional aspects and advantages of the present disclosure will become readily apparent to those skilled in this art from the following detailed description, wherein only illustrative embodiments of the present disclosure are shown and described. As will be realized, the present disclosure is capable of other and different cases, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative cases, in which the principles of the invention are utilized, and the accompanying drawings (also “Figure” and “FIG.” herein), of which:

FIG. 1 illustrates a block diagram of a targeted UV sanitizer system, consistent with certain examples of the present disclosure;

FIG. 2 illustrates an example of operation of detection and tracking techniques identifying surfaces that may be contaminated, consistent with certain examples of the present disclosure;

FIG. 3 illustrates another example of operation of detection and tracking techniques identifying surfaces that may be contaminated, consistent with certain examples of the present disclosure;

FIG. 4 illustrates another example of operation of detection and tracking techniques identifying surfaces that may be contaminated, consistent with certain examples of the present disclosure;

FIG. 5 illustrates an example of active sanitization of surfaces, consistent with certain examples of the present disclosure;

FIG. 6 illustrates another example of active sanitization of surfaces, consistent with certain examples of the present disclosure;

FIG. 7 illustrates a computer control system that is programmed or otherwise configured to implement methods provided herein, consistent with certain examples of the present disclosure;

FIG. 8 illustrates an example method for sanitizing a surface, consistent with certain examples of the present disclosure; and

FIG. 9 illustrates an example method for sanitizing a surface in a confined location, consistent with certain examples of the present disclosure.

### DETAILED DESCRIPTION

In various examples, the systems, the methods, the computer-readable media, or the techniques disclosed herein may significantly reduce the person-to-person spread of contaminants (e.g., bacteria, viruses, pathogens, microorganisms, etc.) in a highly efficient manner and slow down the spread of contaminants that are highly contagious. For example, In some cases, the systems, the methods, the computer-readable media, or the techniques disclosed herein may combine technologies from object detection, motion projection, optical sensing, infrared sensing, or optical beam steering to track motion of one or more subjects (e.g., human subjects). Based on the tracking, a system as disclosed herein may then focus a light beam (e.g., an ultraviolet (UV) beam such as UV-C) on one or more surfaces that those people have contacted or interacted with, such as by passing through/near, touching, or expelling (e.g., coughing, sneezing, wheezing, exhaling, spitting, etc.) contaminants on the surface.

In some embodiments, the systems, the methods, the computer-readable media, or the techniques disclosed herein may be applied to confined spaces (e.g., indoor spaces, outdoor spaces with reduced airflow, etc.) such as spaces, in or around one or more of residential buildings, educational buildings, institutional buildings, assembly buildings, business buildings, mercantile buildings, industrial buildings, storage buildings, or hazardous buildings.

Additional advantages of the systems, the methods, the computer-readable media, or the techniques disclosed herein may include protecting patients and healthcare workers in healthcare (e.g., hospital, clinical, ambulance, etc.) environments, sterilizing products (e.g., food and beverage products) in a production environment, or reducing transmissibility of contaminants in spaces with large public gatherings (e.g., offices, theaters, airports, schools, grocery stores, stadiums, or convention centers). Further, the systems, the methods, the computer-readable media, or the techniques disclosed herein may help in reducing the spread of infectious diseases such as COVID-19, influenza, or other infectious diseases.

As described in further detail below, improved performance of the systems, the methods, the computer-readable media, or the techniques disclosed herein may be characterized by reduction in transmission of contaminants from a surface, reduction in number of contaminants on a surface, reduction in exposure of people or animals to UV radiation, correct prediction of the presence of contaminants on a surface, or other performance metrics.

### Certain Definitions

Unless otherwise defined, all technical terms used herein have the same meaning as commonly understood by one of ordinary skill in the art to which the present subject matter belongs.

As used in this specification and the appended claims, the terms “artificial intelligence,” “artificial intelligence techniques,” “artificial intelligence operation,” and “artificial intelligence algorithm” generally refer to any system or computational procedure that may take one or more actions to enhance or maximize a chance of achieving a goal. The term “artificial intelligence” may include “generative modeling,” “machine learning” (ML), “federated learning,” or “reinforcement learning” (RL).

As used in this specification and the appended claims, the terms “artificial intelligence,” “artificial intelligence techniques,” “artificial intelligence operation,” and “artificial intelligence algorithm” generally refer to any system or computational procedure that may take one or more actions that simulate human intelligence processes for enhancing or maximizing a chance of achieving a goal. The term “artificial intelligence” may include “generative modeling,” “machine learning” (ML), or “reinforcement learning” (RL).

As used in this specification and the appended claims, “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used in this specification and the appended claims, when the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used in this specification and the appended claims, when the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in this specification, “or” is intended to mean an “inclusive or” or what is also known as a “logical OR,” wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “or” unless otherwise stated.

As used in this specification and the appended claims, the indefinite articles “a” or “an,” and the corresponding associated definite articles “the” or “said,” are each intended to mean one or more unless otherwise stated, implied, or physically impossible. Yet further, it should be understood that the expressions “at least one of A and B, etc.,” “at least one of A or B, etc.,” “selected from A and B, etc.” and “selected from A or B, etc.” are each intended to mean either any recited element individually or any combination of two or more elements, for example, any of the elements from the group consisting of “A,” “B,” and “A AND B together,” etc.

As used in this specification and the appended claims “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

### Examples of Machine Learning Methodologies

The systems, the methods, the computer-readable media, or the techniques disclosed herein may use machine learning. In some cases, machine learning may generally involve identifying and recognizing patterns in existing data in order to facilitate making predictions for subsequent data. Machine learning may include processing data using a machine learning model (which may include, for example, a machine learning algorithm). Machine learning, whether analytical or statistical in nature, may provide deductive or abductive inference based on real or simulated data.

The machine learning model may be a trained model. Machine learning (ML) may comprise one or more supervised, semi-supervised, self-supervised, or unsupervised machine learning techniques. For example, an ML model may be a model that is trained through supervised learning (e.g., various parameters are determined as weights or scaling factors).

Training the machine learning model may include, in some cases, selecting one or more untrained data models to train using a training data set. The selected untrained data models may include any type of untrained machine learning models for supervised, semi-supervised, self-supervised, or unsupervised machine learning. The selected untrained data models be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the machine learning model from the selected untrained data models may likewise be selected, such as limits on the machine learning model complexity or limits on the machine learning model refinement past a certain point. The machine learning model may be trained (e.g., via a computer system such as a server) using the training data set. In some cases, a first subset of the training data set may be selected to train the machine learning model. The selected untrained data models may then be trained on the first subset of training data set using appropriate machine learning techniques, based upon the type of machine learning model selected and any conditions specified for training the machine learning model. In some cases, due to the processing power used in training the machine learning model, the selected untrained data models may be trained using additional computing resources (e.g., cloud computing resources). Such training may continue, in some cases, until at least one aspect of the machine learning model is validated and meets selection criteria to be used as a predictive model.

In some cases, one or more aspects of the machine learning model may be validated using a second subset of the training data set (e.g., distinct from the first subset of the training data set) to determine accuracy and robustness of the machine learning model. Such validation may include applying the machine learning model to the second subset of the training data set to make predictions derived from the second subset of the training data. The machine learning model may then be evaluated to determine whether performance is sufficient based upon the derived predictions. The sufficiency criteria applied to the machine learning model may vary depending upon the size of the training data set available for training, the performance of previous iterations of trained models, or user-specified performance objectives. If the machine learning model does not achieve sufficiently adequate performance, additional training may be performed. Additional training may include refinement of the machine learning model or retraining on a different first subset of the training dataset, after which the new machine learning model may again be validated and assessed. When the machine learning model has achieved sufficiently adequate performance (e.g., prediction accuracy above a particular threshold), in some cases, the machine learning model may be stored for present or future use. The machine learning model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of machine learning models may be stored for generating predictions under different sets of input data conditions. In some examples, the machine learning model may be stored in a database (e.g., associated with a server).

ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

The systems, the methods, the computer-readable media, or the techniques disclosed herein may implement one or more computer vision techniques. Computer vision is a field of artificial intelligence that uses computers to interpret and understand the visual world at least in part by processing one or more digital images from cameras and videos. In some cases, computer vision may use deep learning models (e.g., convolutional neural networks). Bounding boxes may be used in object detection techniques within computer vision. Bounding boxes may be annotation markers drawn around objects in an image. Bounding boxes, are often, although not always, may be rectangularly shaped. Bounding boxes may be applied by humans to training data sets. However, bounding boxes may also be applied to images by a trained machine learning that is trained to detect one or more different objects (e.g., humans, hands, faces, cars, etc.).

### Example UV Sanitizer

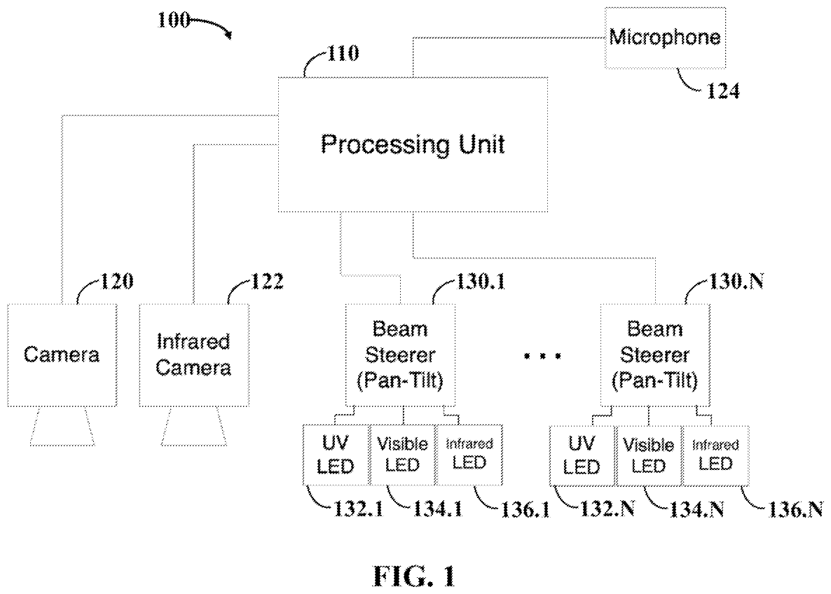

FIG. 1 shows an example block diagram of the camera-assisted active UV-C sanitizer system 100, consistent with certain examples of the present disclosure. The system 100, as illustrated, has 3 main parts: a processing module, a sensing module, and one or multiple UV-C light modules with beam steering (pan and tilt) capability. As illustrated, these modules are included in the system 100 as a processing unit 110, various sensors including a camera 120, an infrared camera 122, and a microphone 124, and a plurality of beam steerers 130.1-130.N including a plurality of UV LEDs 132.1-132.N, a plurality of visible LEDs 134.1-134.N, and a plurality of infrared LEDs 136.1-136.N. The components in FIG. 1 are examples and do not limit the scope of use or functionality of any hardware, software, embedded logic component, or a combination of two or more such components with particular implementations.

Turning first to the computational aspects of the system 100, the system 100 may include the processing unit 110. The processing unit 110 (e.g., a processing or computing system) may include a set of executable instructions that can cause a device to perform or execute any one or more of the methods, the computer-readable media, or the techniques of the present disclosure. While not shown, the processing unit 110 may include one or more processors, a memory, and a storage that communicate with each other, as well as with other components (e.g., the sensors 120-124, the beam steerers 130.1-130.N, etc.), via one or more buses.

The buses may also link a display (not shown), one or more input devices (which may, for example, include a keypad, a keyboard, a mouse, a stylus, etc.; not shown), one or more output devices (not shown), one or more storage device (not shown), or various tangible storage media (not shown). One or more of these elements may interface directly or via one or more interfaces or adaptors to the buses. For example, the various tangible storage media can interface with the bus via storage medium interface. The processing unit 110 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

The processing unit 110 may include one or more processors (e.g., central processing units (CPUs), general purpose graphics processing units (GPGPUs), or quantum processing units (QPUs)) that carry out functions. The processors may optionally contain a cache memory unit for temporary local storage of instructions, data, or computer addresses. The processors included in the processing unit 110 may be configured to assist in execution of computer readable instructions. The processing unit 110 may provide functionality for the components depicted in FIG. 1 as a result of the processors executing non-transitory, processor-executable instructions embodied in one or more tangible computer-readable storage media, such as the memory, the storage, the storage devices, or the storage medium. The computer-readable media included in the processing unit 110 may store software that implements particular operations, and processors that may execute the software. Memory included in the processing unit 110 may read the software from one or more other computer-readable media (such as mass storage devices) or from one or more other sources through a suitable interface, such as network interface. The software of the processing unit 110 may cause processors to carry out one or more processes or one or more operations of one or more processes described or illustrated herein. Carrying out such processes or operations may include defining data structures stored in the memory and modifying the data structures as directed by the software.

In some cases, when the processing unit 110 is connected to a network, the processing unit 110 may communicate with other devices, specifically the sensors 120-124 or the beam steerers 130.1-130.N. In some cases, the processing unit 110 may further communicate, via the network with mobile devices and enterprise systems, distributed computing systems, cloud storage systems, cloud computing systems, or the like. Communications to and from the processing unit 110 may be sent through a network interface (not shown). For example, the network interface may receive incoming communications (such as requests or responses from other devices, e.g., the sensors 120-124 or the beam steerers 130.1-130.N) in the form of one or more packets (such as Internet Protocol (IP) packets) from the network, and the processing unit 110 may store the incoming communications in a memory for processing. The processing unit 110 may similarly store outgoing communications (such as requests or responses to other devices, e.g., the sensors 120-124 or the beam steerers 130.1-130.N) in the form of one or more packets in a memory and communicated to the network from the network interface. Processors of the processing unit 110 may access these communication packets stored in these memories for processing.

Examples of the network interface for enabling communications between the processing unit 110 and the sensors 120-124 or the beam steerers 130.1-130.N may include a network interface card, a modem, and any combination thereof. Examples of a network or network segment for enabling communications between the processing unit 110 and the sensors 120-124 or the beam steerers 130.1-130.N may include a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, or any combinations thereof. The network may employ a wired or a wireless mode of communication. In general, any network topology may be used for enabling communications between the various components of the system 100.

In addition or as an alternative, the processing unit 110 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both. Various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations may be described above generally in terms of their functionality. The various illustrative logical blocks, modules, and circuits described in connection with the examples disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions disclosed herein. A general purpose processor of the processing unit 110 may be a microprocessor, but in the alternative, the processor may be any processor, controller, microcontroller, or state machine. A processor of the processing unit 110 may also be implemented as a combination of computing devices, e.g., a combination of a DSP and a microprocessor, a plurality of microprocessors, one or more microprocessors in conjunction with a DSP core, or any other such configuration. The operations of a method or algorithm described in connection with the examples disclosed herein may be embodied directly in hardware, in a software module executed by one or more processors, or in a combination of the two. A software module included in the processing unit 110 may reside in RAM memory, flash memory, ROM memory, EPROM memory, EEPROM memory, registers, hard disk, a removable disk, a CD-ROM, or any other form of suitable storage medium. An example storage medium may be coupled to the processor such the processor can read information from, and write information to, the storage medium. In the alternative, the storage medium may be integral to the processor. The processor and the storage medium of the processing unit 110 may reside in an ASIC. The ASIC may reside in a user terminal. In the alternative, the processor and the storage medium may reside as discrete components in a user terminal.

The processing unit 110 may receive data from the sensors 120-124. The camera 120 may capture a red-green-blue (RGB) image, a YUV image, or a depth image. The camera 120 may be capable of capturing still images or videos. The camera 120 may be capable of capturing visible light (in the range of about 400 nanometers to about 700 nanometers). The infrared camera 122 may be capable of capturing infrared still images or infrared video. The infrared camera 122 may be capable of capturing infrared light (in the range of about 780 nanometers to about 1000 nanometers). The microphone 124 may be any suitable type of microphone for capturing audio data. The microphone 124 may be capable of converting audio data into electrical signals. The microphone 124 may be one or more of a dynamic microphone, a carbon microphone, a ribbon microphone, a piezoelectric microphone, or any other suitable microphone.

While illustrated as the camera 120, the infrared camera 122, and the microphone 124, the sensors 120-124 may include in addition or in alternative, one or more of thermal sensors and imagers, infrared sensors and imagers, laser ranging sensors, radar sensors, proximity sensors, accelerometers, gyroscopes, pressure sensors, light sensors, ultrasonic sensors, smoke/gas sensors, touch sensors, color sensors, humidity sensors, tilt sensors, photoelectric sensors, vibration sensors, position sensors, pressure sensors, or other suitable sensors.

In general, the sensors 120-124 may be used to identify surfaces for sanitization (e.g., disinfection). For example, the camera 120 may be used to track motion of a person through a space, identifying surfaces the person has contacted (e.g., passed near, touched, coughed on, or otherwise contacted). In another example, the infrared camera 122 may be used to measure the body temperature of a person contacting various surfaces, and, if the person's body temperature is outside a normal level (e.g., outside of about 97° F. and about 99° F.), the person, and the surfaces the person contacted, may be determined as higher risk for having contaminants. In another example, the microphone 124 may be used to track locations of people through a space using audio localization techniques. In another example, the microphone 124 may be used to identify a cough, a sneeze, speaking, wheezing, spitting, or other sounds associated with droplet ejection from a subject's mouth or nose. In another example, the microphone 124 may be used to predict when a space is empty of humans or animals. In another example, motion sensors (which may rely on one or more of pressure sensors, accelerometers, gyroscopes, position sensors, etc.) may determine when an object has been moved or a surface has been contacted. In another example, a proximity sensor may identify when a surface has been contacted (e.g., passed by within a certain distance) or when an area is empty of people or animals. In some cases, the sensors 120-124 may be located in proximity (e.g., physically contacted to or nearby and not contacted to) to the processing unit 110. In some cases, the sensors 120-124 may be located in proximity (e.g., physically contacted to or nearby and not contacted to) to one or more surfaces that may be sanitized.

As described above, the sensors 120-124 may send sensor data (e.g., image data, video data, thermal data, audio data, motion data, location data, position data, etc.) or representations/summaries of the sensor data to the processing unit 110. Using the sensor data, the processing unit may identify the surfaces (e.g., areas, objects, etc.) to sanitize. The processing unit 110 may determine which surfaces to sanitize based on whether or not a subject has contacted (e.g., passed nearby, touched, sneezed on, coughed on, etc.) the surfaces. Determining or predicting whether a subject has contacted a surface may be done using tracking, such as via computer vision techniques (e.g., as described previously). For example, the processing unit 110 may use object detection or tracking techniques (e.g., bounding boxes) to track a subject (e.g., a person or an animal). Tracking a subject may comprise tracking the subject's entire body or tracking a portion of the subject's body. For example, the processing unit 110 may track a subject's hands. In another example, the processing unit 110 may track a subject's face or head. In another example, the processing unit 110 may track a subject's mouth. In another example, the processing unit 110 may track a subject's nose. In another example, the processing unit 110 may track a subject's torso. In some cases, the computer vision techniques for tracking a subject may comprise the use of machine learning. In some cases, the tracking techniques for tracking a subject may not use machine learning.

In some cases, the tracking techniques may include more than tracking where a subject has previously contacted or is currently contacting. Indeed, in some cases, the tracking techniques may predict where a subject is going to contact in the future. For example, the tracking techniques may predict, based on, e.g., a subject's movement trajectory, that the subject will contact a certain surface. In another example, the tracking techniques may predict, based on, e.g., historical data of where subjects have previously contacted, that a subject will contact a certain surface. In another example, the tracking techniques may predict, based on, e.g., modeling or simulating a cough or sneeze, that a subject will have contacted, via droplet expulsion, a certain surface. Predicting where a subject will contact in the future may utilize machine learning techniques. For example, a machine learning model may be trained on video or image data of subjects moving through various spaces to train the machine learning model to predict how subjects will move through a space (e.g., what they will contact in the space) in the future.

In some cases, the processing unit 110 may determine which surfaces to sanitize based on the tracking techniques discussed above. Determining which surface to sanitize based on the tracking of a subject may use artificial intelligence or machine learning. In other cases, determining which surface to sanitize based on the tracking of a subject may not use artificial intelligence or machine learning.

In some cases, the processing unit 110, based on the sensor data received from the sensors 120-124, may determine a contamination risk score for one or more surfaces. The contamination risk score may be based on, for example, a body temperature of a subject (e.g., a person or an animal). The contamination risk score for a surface may be based on, for example, whether or not a subject, who contacted the surface, is wearing a face covering. The contamination risk score for a surface may be based on, for example, whether or not a subject who contacted the surface is coughing, sneezing, wheezing, heaving, talking, shouting, or otherwise expelling droplets from a mouth or nose. The contamination risk score for a surface may be based on, for example, whether or not a subject who contacted the surface has lesions. The contamination risk score for a surface may be based on, for example, whether or not a subject who contacted the surface is exhibiting behavior of having an allergy, disease, virus, or illness. The contamination risk score for a surface may be based on, for example, how long a subject contacted the surface. The contamination risk score for a surface may be based on, for example, how much of a surface was contacted by a subject. The contamination risk score for a surface may be based on, for example, with which body parts (e.g., a mouth may be higher risk than an elbow, etc.) of a subject contacted the surface. The contamination risk score for a surface may be based on, for example, how likely it is a subject contacted the surface. The contamination risk score for a surface may be based on, for example, a physical attribute of the surface (e.g., a porous surface may be higher risk than a less porous surface, etc.). The contamination risk score for a surface may be based on, for example, how frequently the surface is contacted (e.g., a high-contact point like a door handle may be higher risk than a low contact point like a section of flooring in a corner of a room, etc.). The contamination risk score for a surface may be based on, for example, how habitable the surface is to contaminants (e.g., a very hot or very cold surface may be less habitable than a room temperature surface, a surface in direct sunlight may be less habitable than a surface not in direct sunlight, etc.). The contamination risk score for a surface may be based on, for example, how much airflow passes over or by the surface (e.g., a surface in a well-ventilated area may be lower risk than a surface in a less well-ventilated area, etc.). The contamination risk score for a surface may be based on, for example, how often or how long it has been since the surface was manually sanitized (e.g., by a cleaning person, etc.). The contamination risk score for a surface may be based on, for example, any other factors that may determine how much risk a surface has for either (i) being contaminated or (ii) transmitting contamination.

In some cases, based on the contamination risk score, a priority order may be determined by the processing unit 110. The priority order may rank surfaces in descending order of contamination risk score, with surfaces having higher contamination risk score prioritized above surfaces having a lower contamination risk score.

The processing unit 110 may send to the beam steerers 130.1-130.N instructions on where to point a light beam from the light sources (e.g., 132.1-136.1, 132.2-136.2, etc.). The processing unit 110 may send the beam steerers 130.1-130.N the instructions in the determined priority order. The processing unit 110 may send the beam steerers 130.1-130.N the instructions in the order the processing unit 110 identifies surfaces to be sanitized (e.g., in real time). The processing unit 110 may send the beam steerers 130.1-130.N the instructions in any other suitable order. The processing unit 110 may send the beam steerers 130.1-130.N the instructions to point at surfaces where there is not a human or animal at risk of being affected, such as contacted by or in proximity of, the light beam. For example, there may be a safety distance between a person or animal and a surface for sanitization via UV radiation. In some cases, the UV radiation may be radiated onto a non-human living organisms. For example, the UV radiation may be used for pest control, such as for killing insects, rodents, or other pests.

The beam steerers 130.1-130.N may point the light beam towards the surfaces included in the instructions from the processing unit 110. The processing unit 110 may be able to control multiple beam steerers 130.1-130.N at the same time and can target multiple surfaces for sanitization. The operation of each of the beam steerers 130.1-130.N can be independent from the other beam steerers 130.1-130.N.

In some cases, the beam steerers 130.1-130.N steer the light sources 132.1-136.1, . . . , 132.N-136.N. The light sources 132.1-136.1, . . . , 132.N-136.N may emit UV, visible, infrared, or any other type of light that is suitable for sanitizing (e.g., disinfecting) a surface (e.g., killing contaminants on the surface). For example, the light sources 132.1-136.1, . . . , 132.N-136.N may emit UV C light as the light beam. In another example, light sources 132.1-136.1, . . . , 132.N-136.N may emit UV A or UV B light as the light beam.

As described above, In some cases, the light beam may comprise ultraviolet light. In some cases, the wavelength of the light beam is about 100 nanometers to about 400 nanometers. In some cases, the wavelength of the light beam is about 100 nanometers to about 200 nanometers, about 100 nanometers to about 250 nanometers, about 100 nanometers to about 260 nanometers, about 100 nanometers to about 265 nanometers, about 100 nanometers to about 270 nanometers, about 100 nanometers to about 300 nanometers, about 100 nanometers to about 315 nanometers, about 100 nanometers to about 400 nanometers, about 200 nanometers to about 250 nanometers, about 200 nanometers to about 260 nanometers, about 200 nanometers to about 265 nanometers, about 200 nanometers to about 270 nanometers, about 200 nanometers to about 300 nanometers, about 200 nanometers to about 315 nanometers, about 200 nanometers to about 400 nanometers, about 250 nanometers to about 260 nanometers, about 250 nanometers to about 265 nanometers, about 250 nanometers to about 270 nanometers, about 250 nanometers to about 300 nanometers, about 250 nanometers to about 315 nanometers, about 250 nanometers to about 400 nanometers, about 260 nanometers to about 265 nanometers, about 260 nanometers to about 270 nanometers, about 260 nanometers to about 300 nanometers, about 260 nanometers to about 315 nanometers, about 260 nanometers to about 400 nanometers, about 265 nanometers to about 270 nanometers, about 265 nanometers to about 300 nanometers, about 265 nanometers to about 315 nanometers, about 265 nanometers to about 400 nanometers, about 270 nanometers to about 300 nanometers, about 270 nanometers to about 315 nanometers, about 270 nanometers to about 400 nanometers, about 300 nanometers to about 315 nanometers, about 300 nanometers to about 400 nanometers, or about 315 nanometers to about 400 nanometers. In some cases, the wavelength of the light beam is greater than about 100 nanometers, greater than about 200 nanometers, greater than about 250 nanometers, greater than about 260 nanometers, greater than about 265 nanometers, greater than about 270 nanometers, greater than about 300 nanometers, greater than about 315 nanometers, or greater than about 400 nanometers. In some cases, the wavelength of the light beam is greater than about 100 nanometers, greater than about 200 nanometers, greater than about 250 nanometers, greater than about 260 nanometers, greater than about 265 nanometers, greater than about 270 nanometers, greater than about 300 nanometers, or greater than about 315 nanometers. In some cases, the wavelength of the light beam is greater than about 200 nanometers, greater than about 250 nanometers, greater than about 260 nanometers, greater than about 265 nanometers, greater than about 270 nanometers, greater than about 300 nanometers, greater than about 315 nanometers, or greater than about 400 nanometers. In some cases, the wavelength of the light beam is less than about 100 nanometers, less than about 200 nanometers, less than about 250 nanometers, less than about 260 nanometers, less than about 265 nanometers, less than about 270 nanometers, less than about 300 nanometers, less than about 315 nanometers, or less than about 400 nanometers. In some cases, the wavelength of the light beam is less than about 100 nanometers, less than about 200 nanometers, less than about 250 nanometers, less than about 260 nanometers, less than about 265 nanometers, less than about 270 nanometers, less than about 300 nanometers, or less than about 315 nanometers. In some cases, the wavelength of the light beam is less than about 200 nanometers, less than about 250 nanometers, less than about 260 nanometers, less than about 265 nanometers, less than about 270 nanometers, less than about 300 nanometers, less than about 315 nanometers, or less than about 400 nanometers.

In some cases, the light beam may be omni-directional. In some cases, the light beam may be collimated. In some cases, the light beam may be directional. In some cases, the light beam has a beam spread of about 0 degrees to about 180 degrees. In some cases, the light beam has a beam spread of about 0 degrees to about 1 degree, about 0 degrees to about 3 degrees, about 0 degrees to about 5 degrees, about 0 degrees to about 10 degrees, about 0 degrees to about 15 degrees, about 0 degrees to about 25 degrees, about 0 degrees to about 45 degrees, about 0 degrees to about 60 degrees, about 0 degrees to about 90 degrees, about 0 degrees to about 120 degrees, about 0 degrees to about 180 degrees, about 1 degree to about 3 degrees, about 1 degree to about 5 degrees, about 1 degree to about 10 degrees, about 1 degree to about 15 degrees, about 1 degree to about 25 degrees, about 1 degree to about 45 degrees, about 1 degree to about 60 degrees, about 1 degree to about 90 degrees, about 1 degree to about 120 degrees, about 1 degree to about 180 degrees, about 3 degrees to about 5 degrees, about 3 degrees to about 10 degrees, about 3 degrees to about 15 degrees, about 3 degrees to about 25 degrees, about 3 degrees to about 45 degrees, about 3 degrees to about 60 degrees, about 3 degrees to about 90 degrees, about 3 degrees to about 120 degrees, about 3 degrees to about 180 degrees, about 5 degrees to about 10 degrees, about 5 degrees to about 15 degrees, about 5 degrees to about 25 degrees, about 5 degrees to about 45 degrees, about 5 degrees to about 60 degrees, about 5 degrees to about 90 degrees, about 5 degrees to about 120 degrees, about 5 degrees to about 180 degrees, about 10 degrees to about 15 degrees, about 10 degrees to about 25 degrees, about 10 degrees to about 45 degrees, about 10 degrees to about 60 degrees, about 10 degrees to about 90 degrees, about 10 degrees to about 120 degrees, about 10 degrees to about 180 degrees, about 15 degrees to about 25 degrees, about 15 degrees to about 45 degrees, about 15 degrees to about 60 degrees, about 15 degrees to about 90 degrees, about 15 degrees to about 120 degrees, about 15 degrees to about 180 degrees, about 25 degrees to about 45 degrees, about 25 degrees to about 60 degrees, about 25 degrees to about 90 degrees, about 25 degrees to about 120 degrees, about 25 degrees to about 180 degrees, about 45 degrees to about 60 degrees, about 45 degrees to about 90 degrees, about 45 degrees to about 120 degrees, about 45 degrees to about 180 degrees, about 60 degrees to about 90 degrees, about 60 degrees to about 120 degrees, about 60 degrees to about 180 degrees, about 90 degrees to about 120 degrees, about 90 degrees to about 180 degrees, or about 120 degrees to about 180 degrees. In some cases, the light beam has a beam spread of greater than about 0 degrees, greater than about 1 degree, greater than about 3 degrees, greater than about 5 degrees, greater than about 10 degrees, greater than about 15 degrees, greater than about 25 degrees, greater than about 45 degrees, greater than about 60 degrees, greater than about 90 degrees, greater than about 120 degrees, or greater than about 180 degrees. In some cases, the light beam has a beam spread of greater than about 0 degrees, greater than about 1 degree, greater than about 3 degrees, greater than about 5 degrees, greater than about 10 degrees, greater than about 15 degrees, greater than about 25 degrees, greater than about 45 degrees, greater than about 60 degrees, greater than about 90 degrees, or greater than about 120 degrees. In some cases, the light beam has a beam spread of greater than about 1 degree, greater than about 3 degrees, greater than about 5 degrees, greater than about 10 degrees, greater than about 15 degrees, greater than about 25 degrees, greater than about 45 degrees, greater than about 60 degrees, greater than about 90 degrees, greater than about 120 degrees, or greater than about 180 degrees. In some cases, the light beam has a beam spread of less than about 0 degrees, less than about 1 degree, less than about 3 degrees, less than about 5 degrees, less than about 10 degrees, less than about 15 degrees, less than about 25 degrees, less than about 45 degrees, less than about 60 degrees, less than about 90 degrees, less than about 120 degrees, or less than about 180 degrees. In some cases, the light beam has a beam spread of less than about 0 degrees, less than about 1 degree, less than about 3 degrees, less than about 5 degrees, less than about 10 degrees, less than about 15 degrees, less than about 25 degrees, less than about 45 degrees, less than about 60 degrees, less than about 90 degrees, or less than about 120 degrees. In some cases, the light beam has a beam spread of less than about 1 degree, less than about 3 degrees, less than about 5 degrees, less than about 10 degrees, less than about 15 degrees, less than about 25 degrees, less than about 45 degrees, less than about 60 degrees, less than about 90 degrees, less than about 120 degrees, or less than about 180 degrees.

In some cases, the processing unit 110 may include, in instructions to the beam steerers 130.1-130.N, a sanitization time or a strength of a light beam. The sanitization time or the strength of the light beam may be based at least in part on the contamination risk score. For example, if the contamination risk score for a first surface is greater than the contamination risk score for a second surface, then one or both of the sanitization time or the strength of the light beam corresponding to the first surface may be greater than for the second surface. Strength of the light beam may be based on illuminance (e.g., in footcandles, lux, lumens, etc.) of the light beam. Sanitization time may be an amount of time the light beam shines light on a surface. In some cases, sanitization time may be as short as fractions of a second, while in other cases the sanitization time may be longer, e.g., one or more seconds, one or more minutes, one or more hours, etc. In some cases, by increasing the strength of the light beam, the sanitization time for a given surface may be reduced (and vice-versa).

In some cases, the system 100 uses the light sources 132.1-136.1, . . . , 132.N-136.N to provide highly directional and controllable light beams (e.g., highly collimated UV-C light) that may be steered, using the beam steerers 130.1-130.N, towards surfaces that have a contamination risk. The system 100 may, in some cases, exploit the power of AI and use an advanced tracking techniques with the processing unit 110 to detect people while they are moving, project their paths, and focus the light beams on the surfaces they have contacted to sanitize them before another person contacts them. The system 100 may, in some cases, have the capability to kill contaminants that are spread from a sick person in the form of aerosols (e.g., by predicting or simulating a likely contamination area of the aerosols). Deploying the system 100 at hospitals, schools, transportation hubs such as airports, stores, or offices may enable people e to continue in their daily routines while the system 100 sanitizes spaces between people and reduces contaminant transmission risk among them.

### Example Operation of Detection and Tracking Techniques

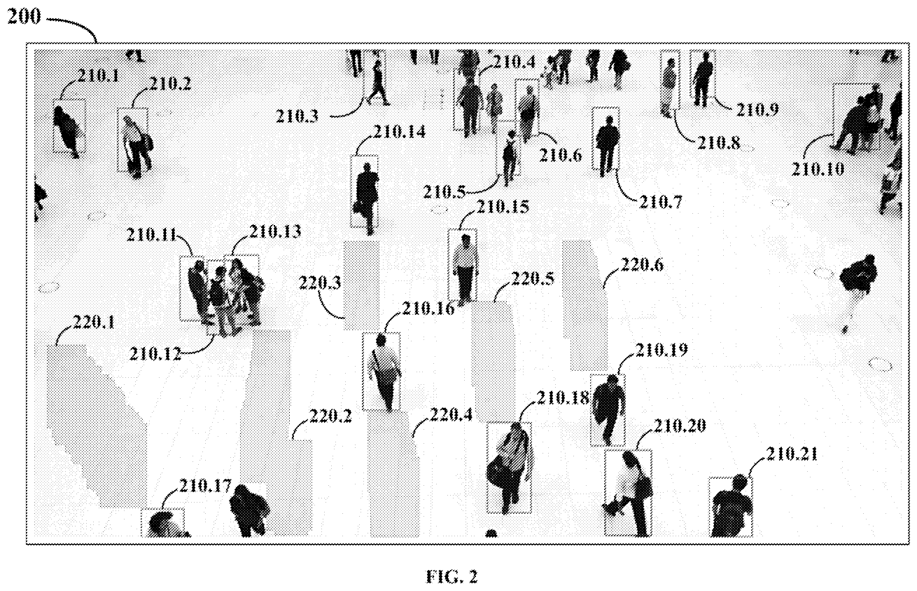

FIG. 2 illustrates an example 200 of operation of detection and tracking techniques identifying surfaces that may be contaminated in an image, consistent with certain examples of the present disclosure. The operation of the detection and tracking techniques of the example 200 may be implemented using computer processing (e.g., computer vision or machine learning techniques). The operation of the detection and tracking techniques of the example 200 may be implemented using one or more components of the system 100, such as the processing unit 110 or the sensors 120-124 (e.g., the camera 120).

As illustrated, the example 200 includes a plurality of subjects (humans, as illustrated) moving through a space. The space may be a confined space. The space may be indoors. The space may be a public space. The space may be a large public space, such as a train platform, airport terminal, shopping mall, or supermarket. The example 200 includes a plurality of bounding boxes 210.1-210.21 that are superimposed on the image. As illustrated, a bounding box may encompass image data of one of the subjects. Accordingly, a bounding box may annotate the presence, approximate location, and approximate dimensions of one of the subjects. As illustrated, not all the subjects are encompassed by one of the bounding boxes 210.1-210.21. In some cases, by further training a computer vision algorithm to track subjects, the computer vision algorithm will be more capable of identifying subjects (e.g., via the plurality of bounding boxes 210.1-210.21). While illustrated as using the bounding boxes 210.1-210.21, the detection and tracking techniques may use, in addition or in alternative, any object detection annotation techniques for detecting and tracking subjects, such as semantic segmentation, instance segmentation, polygon annotation, non-polygon annotation, landmarking, or 3D cuboids.

In the example 200, by tracking the subjects, the detection and tracking techniques may predict surfaces that may be contaminated. The detection and tracking techniques may use edge or cloud processing to run the detection and tracking techniques. As illustrated, the detection and tracking techniques may detect the subjects (via the bounding boxes 210.1-210.21) and a corresponding plurality of movement paths 220.1-220.6. The plurality of movement paths 220-220.6 may be annotated on the image of the example 200 using a polygon representation of the approximate path traversed by a subject. The movement paths 220-220.6 may therefore represent surfaces that are predicted to have contaminants deposited thereon. In some cases, the movement paths 220-220.6 represent the entire area of the surface that is predicted to have contaminants deposited thereon. In some cases, the movement paths 220-220.6 represent an area of the surface that is predicted to have more than a threshold amount of contaminants deposited thereon. In some cases, the movement paths 220-220.6 represent an area of the surface that has more than a threshold likelihood of having contaminants deposited thereon. In some cases, the movement paths 220-220.6 represent an area of the surface that has more than a threshold contaminant risk score associated therewith.

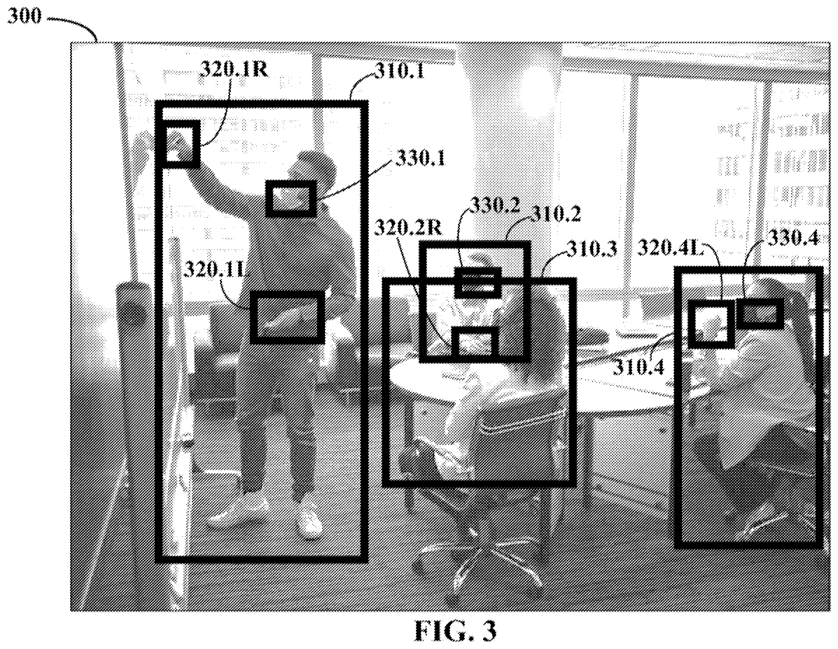

FIG. 3 illustrates another example 300 of operation of detection and tracking techniques identifying surfaces that may be contaminated in an image, consistent with certain examples of the present disclosure. The operation of the detection and tracking techniques of the example 300 may be implemented using machine learning or computer vision techniques. The operation of the detection and tracking techniques of the example 300 may be implemented using one or more components of the system 100, such as the processing unit 110 or the sensors 120-124 (e.g., the camera 120). The detection and tracking techniques of the example 300 may be the same as or similar to the detection and tracking techniques described with respect to FIG. 2.

In FIG. 3, the example 300 includes a plurality of bounding boxes 310.1-310.4 that encompass image data corresponding to subjects. Further, the example 300 includes a plurality of bounding boxes 320.1L, 320.1R 320.2R, and 320.4L that encompass image data corresponding to the hands of the subjects. Detecting and tracking the hands of the subjects may be advantageous in predicting if a surface is infected as the hands of subjects may often transmit contaminants to surfaces. Further, the example 300 includes a plurality of bounding boxes 330.1, 3320.2, and 330.4 that encompass image data corresponding to masks of the subjects. Detecting the present of masks on the subjects may be advantageous in predicting if a surface is infected as masks may reduce transmission of contaminants from the mouths and noses of subjects to surfaces. While bounding boxes are illustrated in the example 300 as encompassing image data corresponding to the subjects, the hands of the subjects, and the masks of the subjects, further or alternative object annotations (e.g., bounding boxes, semantic segmentation, instance segmentation, polygon annotation, non-polygon annotation, landmarking, or 3D cuboids) may be used to encompass image data corresponding to one or more of: the subjects, the hands of the subjects, the masks of the subjects, the heads of the subjects, faces of the subjects, mouths of the subjects, noses of the subjects, aerosol projections of the subjects, etc.

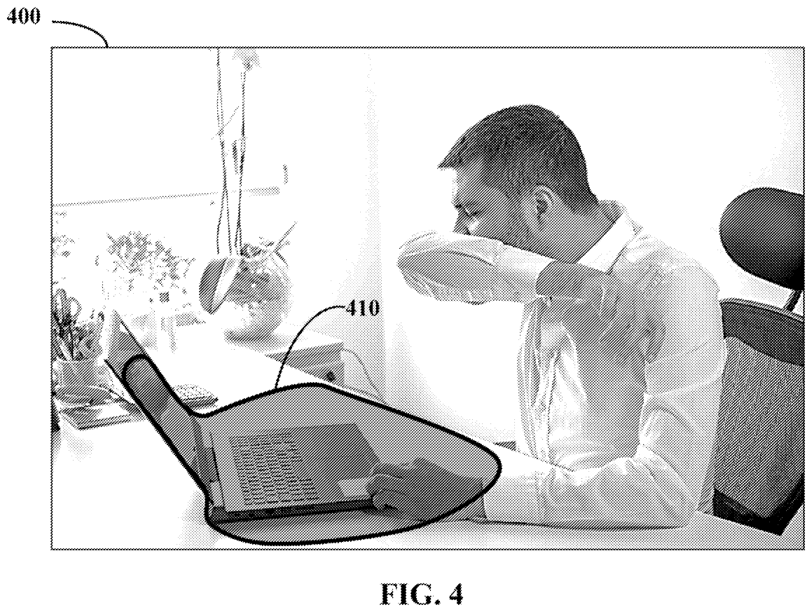

FIG. 4 illustrates another example 400 of operation of detection and tracking techniques identifying surfaces that may be contaminated in an image, consistent with certain examples of the present disclosure. The operation of the detection and tracking techniques of the example 400 may be implemented using machine learning techniques (e.g., computer vision techniques). The operation of the detection and tracking techniques of the example 400 may be implemented using one or more components of the system 100, such as the processing unit 110 or the sensors 120-124 (e.g., the camera 120 or the microphone 124). The detection and tracking techniques of the example 400 may be the same as or similar to the detection and tracking techniques described with respect to FIG. 2 or FIG. 3.

In FIG. 4, the example 400 includes a non-polygon annotation 410 that encompass image data corresponding to a surface identified for decontamination in response to a cough or sneeze by a subject. The non-polygon annotation 410 may be based on a predicted or a simulated dispersion of contaminants (e.g., aerosol, droplets, etc.) from the sneeze or the cough. In some cases, the predicted or the simulated dispersion of the contaminants may be based on image or video data (e.g., provided by a camera, such as the camera 120). In some cases, the predicted or the simulated dispersion of the contaminants may be based on audio data (e.g., provided by a microphone, such as the microphone 124).

The non-polygon annotation 410 may represent the surface that is predicted to have contaminants deposited thereon. In some cases, the non-polygon annotation 410 represents the entire area of the surface that is predicted to have contaminants deposited thereon. In some cases, the non-polygon annotation 410 represents an area of the surface that is predicted to have more than a threshold amount of contaminants deposited thereon. In some cases, the non-polygon annotation 410 represents an area of the surface that has more than a threshold likelihood of having contaminants deposited thereon. In some cases, the non-polygon annotation 410 represents an area of the surface that has more than a threshold contaminant risk score associated therewith. While illustrated as using the non-polygon annotation 410, the detection and tracking techniques may use, in addition or in alternative, any object detection annotation techniques for representing the surface that is predicted to have contaminants deposited thereon, such as bounding boxes, semantic segmentation, instance segmentation, polygon annotation, landmarking, or 3D cuboids.

### Example Active Sanitization of Surfaces

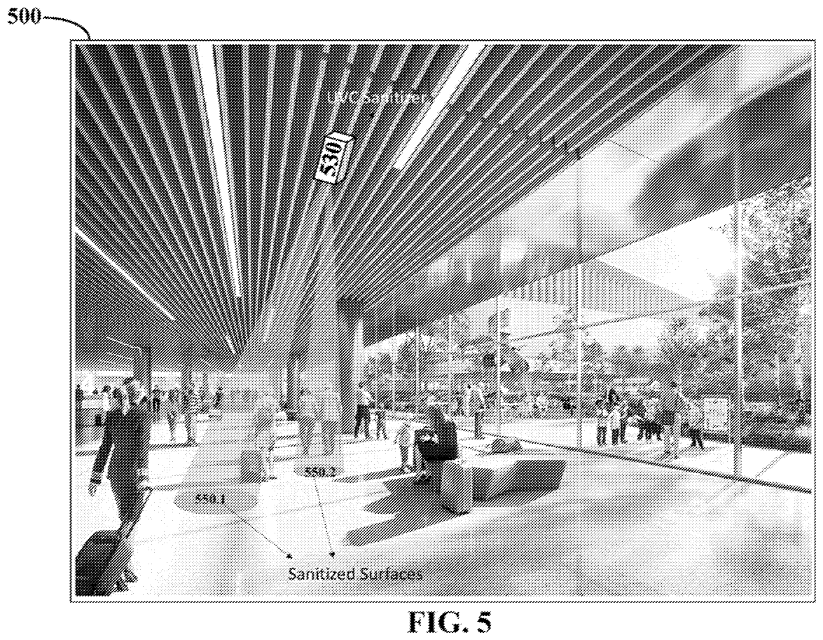

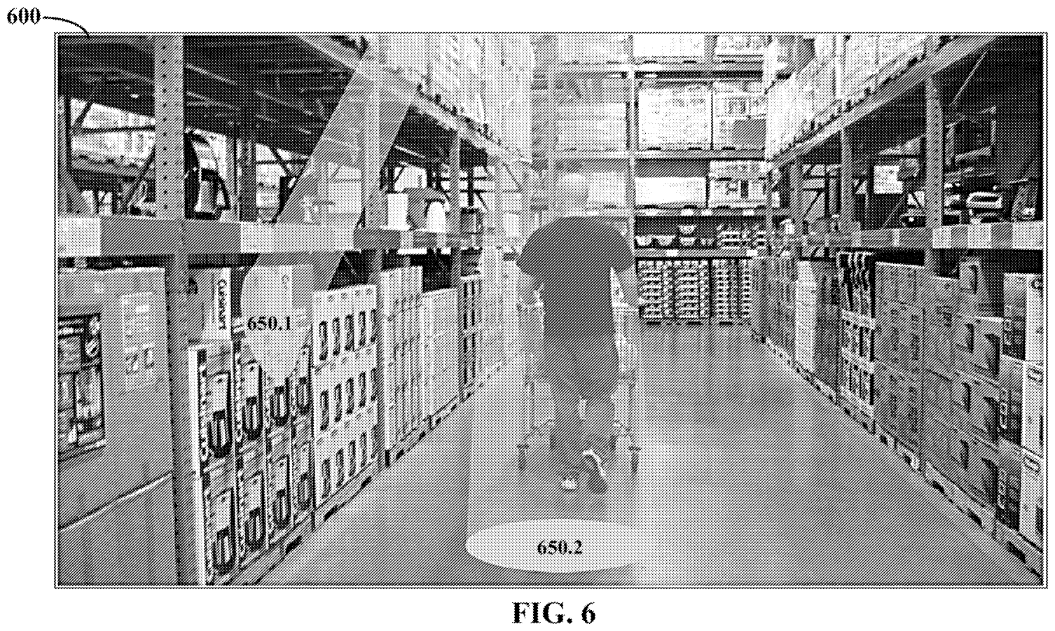

FIGS. 5 and 6 each illustrate an examples 500 and 600, respectively of active sanitization of surfaces, consistent with certain examples of the present disclosure. The operation of the active sanitization of surfaces of the examples 500 and 600 may be implemented using computer processing techniques such as machine learning techniques or computer vision techniques. The operation of active sanitization of surfaces of the example 500 and 600 may be implemented using one or more components of the system 100, such as the processing unit 110 or the sensors 120-124 (e.g., the camera 120).

As illustrated, the examples 500 and 600 include subjects (humans, as illustrated) moving through a space. The space may be a confined space. The space may be indoors. The space may be a public space (e.g., an airport as in the example 500, a store as in the example 600, etc.). The example 500 includes a light source 530 that may include one or more elements of the system 100. For example, the light source 530 may include a beam steerer (e.g., the beam steerers 130.1-130.N). In another example, the light source 530 may include one or more LEDs (e.g., the UV LED 132.1-132.N, the visible LED 134.1-134.N, the infrared LED 136.1-136.N). In another example, the light source 530 may include a computer system or processors (e.g., the processing unit 110). In another example, the light source 530 may include one or more sensors (e.g., the sensors 120-124).

As illustrated, the light source 530 may emit light beams used for sanitizing (e.g., disinfecting) one or more surfaces. The light beams may be ultraviolet light (e.g., UV-A light, UV-B light, UV-C light, etc), such as described with respect to FIG. 1. The light source 530 may direct (e.g., via beam steerers, such as the beam steerers 130.1-130.N) the light beams on surfaces such as flooring, walls, ceiling, seating, doors, windows, furniture, objects, or any other non-human or non-animal surfaces. The light source 530 may direct the light beams on surfaces to sanitize the surfaces (e.g., based on a contamination risk score). For example, sanitized surfaces 550.1 and 550.2 may correspond to where people in the airport have previously contacted (e.g., walked on). As illustrated, the light source 530 may emit light beams while subjects are in the same room as the surfaces being sanitized. As illustrated, the light sources 530 may steer the light beams towards surfaces that are a safe distance from subjects. The safe distance may be a minimum distance (e.g., 1 foot, 5 feet, 10 feet, 30 feet, etc.). Similarly, in the example 600, light beams may sanitize surfaces 650.1 and 650.2 that correspond to where a person has contacted (e.g., walked on or touched) without the light beams radiating light onto the person (or any other subjects).

### Example Computer Systems

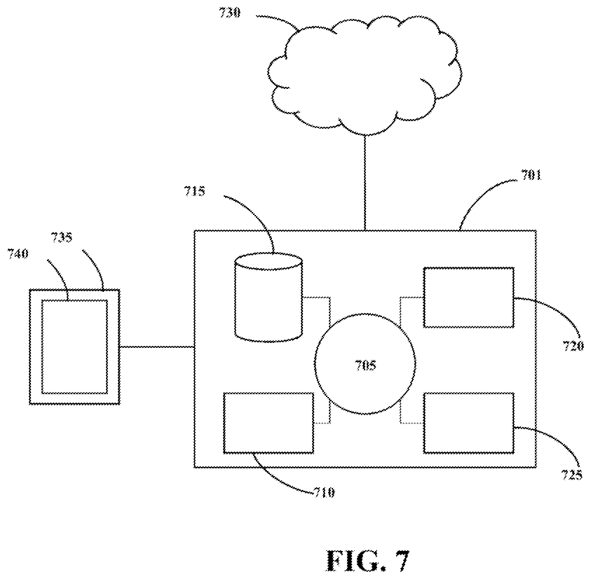

FIG. 7 shows a computer system 701 that is programmed or otherwise configured to operate the systems, the methods, the computer-readable media, or the techniques disclosed herein (such as systems or methods for sanitizing surfaces, disclosed herein). The computer system 701 may be the same as, similar to, an implementation of, or included in the processing unit 110 of FIG. 1.

The computer system 701 can regulate various aspects of the present disclosure. The computer system 701 can be an electronic device of a user or a computer system that is remotely located with respect to the electronic device. The electronic device can be a mobile electronic device.

The computer system 701 includes a central processing system (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage system 715 (e.g., hard disk), communication interface 720 (e.g., network adapter) for communicating with one or more other systems, and peripheral devices 725, such as cache, other memory, data storage or electronic display adapters. The memory 710, storage system 715, interface 720 and peripheral devices 725 are in communication with the CPU 705 through a communication bus (solid lines), such as a motherboard. The storage system 715 can be a data storage system (or data repository) for storing data. The computer system 701 can be operatively coupled to a computer network (“network”) 730 with the aid of the communication interface 720. The network 730 can be the Internet, an internet or extranet, or an intranet or extranet that is in communication with the Internet. The network 730 in some cases is a telecommunication or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage system 715 can store files, such as drivers, libraries and saved programs. The storage system 715 can store user data, e.g., user preferences and user programs. The computer system 701 in some cases can include one or more additional data storage systems that are external to the computer system 701, such as located on a remote server that is in communication with the computer system 701 through an intranet or the Internet.

The computer system 701 can communicate with one or more remote computer systems through the network 730. For example, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as disclosed herein can be implemented by way of machine (e.g., computer processor) executable code stored on an electronic storage location of the computer system 701, such as, for example, on the memory 710 or electronic storage system 715. The machine executable or machine readable code can be provided in the form of software. During use, the code can be executed by the processor 705. In some cases, the code can be retrieved from the storage system 715 and stored on the memory 710 for ready access by the processor 705. In some situations, the electronic storage system 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code or associated data that is carried on or embodied in a type of machine readable medium. Machine-executable code can be stored on an electronic storage system, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code (e.g., computer-readable media), may take many forms, including but not limited to, a tangible storage medium, a carrier wave medium or physical transmission medium. Non-volatile storage media include, for example, optical or magnetic disks, such as any of the storage devices in any computers or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer readable media may be involved in carrying one or more sequences of one or more instructions to a processor for execution.

The computer system 701 can include or be in communication with an electronic display 735 that comprises a user interface (UI) 740. Examples of UI's include, without limitation, a graphical user interface (GUI) and web-based user interface.

The systems, the methods, the computer-readable media, or the techniques of the present disclosure can be implemented by way of one or more algorithms. An algorithm can be implemented by way of software upon execution by the central processing system 705.

### Example Method

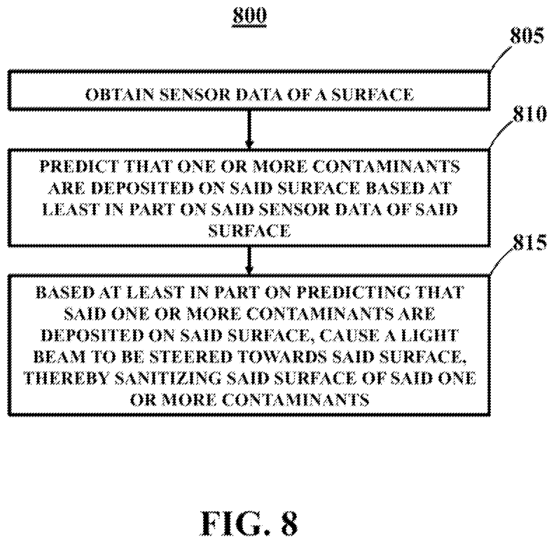

FIG. 8 illustrates an example method 800 for sanitizing a surface, consistent with certain examples of the present disclosure. The method 800 may include obtaining sensor data of the surface (block 805); predicting that one or more contaminants are deposited on the surface based at least in part on the sensor data of the surface (block 810); and based at least in part on predicting that the one or more contaminants are deposited on the surface, causing a light beam to be steered towards the surface, thereby sanitizing the surface of the one or more contaminants (block 815). The method 800 may implement one or more of the systems, methods, computer-readable media, or techniques that are disclosed herein.

In some cases, the method 800 may begin with obtaining sensor data of the surface at the block 805. In some cases, the sensor data is collected by one or more sensors. In some cases, the sensors may comprise a thermal sensor, image sensor, infrared sensor, laser ranging sensor, audio sensor, a radar sensor, or a combination thereof. In some cases, the audio sensor may be a microphone. In some cases, the image sensor may be a camera. In some cases, the camera may produce a red-green-blue (RGB) image, a YUV image, or a depth image. In some cases, the surface is in a public area comprising a plurality of subjects. The sensor data and sensors may be the same as or similar to the sensor data and the sensors shown and described with respect to FIG. 1 (e.g., with reference to the sensors 120-124).

In some cases, the method 800 may comprise predicting that one or more contaminants are deposited on the surface based at least in part on the sensor data of the surface at the block 810. In some cases, predicting that the one or more contaminants are deposited on the surface may comprise a use of a machine learning model. In some cases, predicting that the one or more contaminants are deposited on the surface comprises a use of a computer vision model. In some cases, predicting that the one or more contaminants are deposited on the surface comprises tracking a subject to predict that the subject has deposited the one or more contaminants on the surface. In some cases, the tracking the subject comprises tracking the subject using a bounding box. In some cases, the tracking the subject comprises tracking a body part of the subject or tracking a spread of an aerosol or a droplet of the subject. In some cases, the body part is a hand. In some cases, the spread of the aerosol or the droplet is produced by a cough, sneeze, wheeze, heave, utterance, or expulsion of air from a mouth of nose of the human subject. In some cases, tracking the spread of the aerosol or the droplet of the subject comprises simulating the spread of the aerosol or the droplet of the subject. In some cases, the one or more contaminants comprise microorganisms. In some cases, the microorganisms comprise pathogens. In some cases, the pathogens comprise bacteria or viruses. In some cases, predicting that one or more contaminants are deposited on the surface based at least in part on the sensor data of the surface comprises: (i) assigning a score for the surface; and (ii) adjusting a sanitization time and a strength of the light beam based at least in part on the score. In some cases, the score is based at least in part on a determination of the surface comprising at least one subject at risk of illness. In some cases, the determination is based at least in part on a body temperature of the at least one subject. The predicting that one or more contaminants are deposited on the surface based at least in part on the sensor data of the surface may be the same as or similar to the predicting described with respect to FIG. 1 (e.g., with reference to the processing unit 110). The predicting that one or more contaminants are deposited on the surface based at least in part on the sensor data of the surface may be the same as or similar to the examples 500 and 600 shown and described in FIGS. 2-6.

In some cases, the method 800 may comprise based at least in part on predicting that the one or more contaminants are deposited on the surface, causing a light beam to be steered towards the surface, thereby sanitizing (e.g., disinfecting) the surface of the one or more contaminants at the block 815. In some cases, prior to the block 815, the method 800 may further comprise calculating a priority for the surface, wherein the priority is based at least in part on a contamination risk of the surface. In some cases, the contamination risk of the surface is determined based at least in part on a body temperature of a subject. In some cases, the contamination risk is determine based at least in part on a presence or absence of a face covering worn by a subject. In some cases, causing the light beam to be steered towards the surface is performed in accordance with the priority. In some cases, the light beam is an ultraviolet beam. In some cases, causing the light beam to be steered towards the surface at the block 815 comprises: (i) generating a control signal based at least in part on the surface; (ii) providing the control signal to a beam steerer, causing the beam steerer to be directed towards the surface; and (iii) activating a source of the light beam. In some cases, a wavelength of the light beam is between 200 and 300 nm. In some cases, the wavelength is between 250 and 300 nm. In some cases, the wavelength is between 260 and 270 nm. The light beam may be the same as or similar to the light beam and light sources shown and described with respect to FIG. 1 (e.g., with reference to the beam steerers 130.1-130.N or the light sources 132.1-136.1, . . . , 132.N-136.N). The light beam may be the same as or similar to the light beam shown and described with respect to FIGS. 5 and 6.

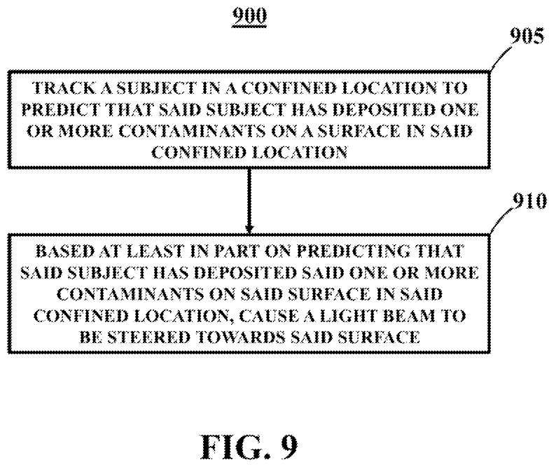

FIG. 9 illustrates an example method 900 for sanitizing a surface in a confined location, consistent with certain examples of the present disclosure. The method 900 may include tracking a subject in the confined location to predict that the subject has deposited one or more contaminants on the surface in the confined location (block 905); and based at least in part on predicting that the subject has deposited the one or more contaminants on the surface in the confined location, causing a light beam to be steered towards the surface (block 910). In some cases, one or more operation of the method 900 may be performed while the subject or another subject is present in the confined location. The method 900 may implement one or more of the systems, methods, computer-readable media, or techniques that are disclosed herein. The method 900 may include operations that are the same as or similar to the operations shown and described in the method 800 of FIG. 8.

### Additional Considerations

While various embodiments of the invention have been shown and disclosed herein, it will be obvious to those skilled in the art that such embodiments are provided by way of example only. Numerous variations, changes, and substitutions may occur to those skilled in the art without departing from the invention. It should be understood that various alternatives to the embodiments of the invention disclosed herein may be employed.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases.

It should be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, hardware modules may encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information). Elements that are described as being coupled and or connected may refer to two or more elements that may be (e.g., direct physical contact) or may not be (e.g., electrically connected, communicatively coupled, etc.) in direct contact with each other, but yet still cooperate or interact with each other.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

While preferred embodiments of the present invention have been shown and disclosed herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention disclosed herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for sanitizing a surface, comprising:
(a) obtaining, at one or more processors, sensor data of said surface;
(b) predicting, by said one or more processors, that one or more contaminants are deposited on said surface based at least in part on said sensor data of said surface; and
(c) based at least in part on predicting that said one or more contaminants are deposited on said surface at (b), causing, by said one or more processors, a light beam to be steered towards said surface, thereby sanitizing said surface of said one or more contaminants.

2. The method of claim 1, wherein predicting that said one or more contaminants are deposited on said surface at (b) comprises a use of a machine learning model.

3. The method of claim 2, wherein predicting that said one or more contaminants are deposited on said surface at (b) comprises a use of a computer vision model.

4. The method of claim 1, wherein said light beam is an ultraviolet beam.

5. The method of claim 1, wherein predicting that said one or more contaminants are deposited on said surface at (b) comprises tracking a subject to predict that said subject has deposited said one or more contaminants on said surface.

6. The method of claim 5, wherein said tracking said subject comprises tracking said subject using a bounding box.

7. The method of claim 5, wherein said tracking said subject comprises tracking a body part of said subject or tracking a spread of an aerosol or a droplet of said subject.

8. The method of claim 7, wherein said body part is a hand.

9. The method of claim 7, wherein said spread of said aerosol or said droplet is produced by a cough, sneeze, wheeze, heave, utterance, or expulsion of air from a mouth of nose of said human subject.

10. The method of claim 7 wherein tracking said spread of said aerosol or said droplet of said subject comprises simulating said spread of said aerosol or said droplet of said subject.

11. The method of claim 1, further comprising, prior to (c), calculating a priority for said surface, wherein said priority is based at least in part on a contamination risk of said surface.

12. The method of claim 11, wherein said contamination risk of said surface is determined based at least in part on a body temperature of a subject.

13. The method of claim 11, wherein said contamination risk is determine based at least in part on a presence or absence of a face covering worn by a subject.

14. The method of claim 11, wherein causing said light beam to be steered towards said surface at (c) is performed in accordance with said priority.

15. The method of claim 1, wherein said one or more contaminants comprise microorganisms.

16. The method of claim 15, wherein said microorganisms comprise pathogens.

17. The method of claim 16, wherein said pathogens comprise bacteria or viruses.

18. The method of claim 1, wherein causing said light beam to be steered towards said surface at (c) comprises:
(i) generating a control signal based at least in part on said surface;
(ii) providing said control signal to a beam steerer, causing said beam steerer to be directed towards said surface; and
(iii) activating a source of said light beam.

19. The method of claim 1, wherein said surface is in a public area comprising a plurality of subjects.

20. The method of claim 1, wherein predicting that one or more contaminants are deposited on said surface based at least in part on said sensor data of said surface comprises:
(i) assigning a score for said surface; and
(ii) adjusting a sanitization time and a strength of said light beam based at least in part on said score.

21. The method of claim 20, wherein said score is based at least in part on a determination of said surface comprising at least one subject at risk of illness.

22. The method of claim 21, wherein said determination is based at least in part on a body temperature of said at least one subject.

23. The method of claim 1, wherein the sensor data is collected by one or more sensors that comprise a thermal sensor, image sensor, infrared sensor, laser ranging sensor, audio sensor, a radar sensor or imager, or a combination thereof.

24. The method of claim 23, wherein the audio sensor is a microphone.

25. The method of claim 23, wherein the image sensor is a camera.

26. The method of claim 25, wherein the camera produces a red-green-blue (RGB) image, a YUV image, or a depth image.

27. The method of claim 1, wherein a wavelength of said light beam is between 200 and 300 nm.

28. The method of claim 27, wherein said wavelength is between 250 and 300 nm.

29. The method of claim 28, wherein said wavelength is between 260 and 270 nm.

30. A method for sanitizing a surface in a confined location, comprising:
(a) tracking, by one or more processors, a subject in said confined location to predict that said subject has deposited one or more contaminants on said surface in said confined location; and
(b) based at least in part on predicting that said subject has deposited said one or more contaminants on said surface in said confined location, causing, by the one or more processors, a light beam to be steered towards said surface.

31. The method of claim 30, wherein (b) is performed while said subject or another subject is present in said confined location.

32. A system for sanitizing a surface in a confined location, comprising:
(a) a light source configured to provide a light beam having a wavelength or wavelength range sufficient to sanitize said surface;
(b) one or more sensors configured to track a subject in said confined location; and
(c) one or more computer processors operatively coupled to said light source and said one or more sensors, wherein said one or more computer processors are individually or collectively configured to:
(i) predict, based at least in part on sensor data from said one or more sensors, that said subject has deposited one or more contaminants on said surface in said confined location; and
(ii) based at least in part on predicting that said subject has deposited said one or more contaminants on said surface in said confined location, cause said light source to be steered to direct said light beam towards said surface.
